(19) United States
(12) Patent Application Publication
(10) Pub. No.: US 2026/0334744 A1
(43) Pub. Date: Oct. 29, 2026

(54) SYSTEM AND METHOD FOR AUTONOMOUS ARTIFICIAL INTELLIGENCE-BASED (AI-BASED) CLEANING OF DIVERSE SURFACES

(71) Applicant: RMATICS SOLUTIONS LLP, HYDERABAD (IN)

(72) Inventor: RAJASEKHAR REDDY KALAKOTA, HYDERABAD (IN)

(21) Appl. No.: 19/231,624

(22) Filed: Jun. 9, 2025

(30) Foreign Application Priority Data
Apr. 23, 2025 (IN) 202541038921

(51) Int. Cl. A47L 11/40; A61L 2/10; A61L 2/24; B25J 9/00; G05D 1/435; G05D 1/648; G05D 109/12; G06V 10/82; G06V 20/58

(52) U.S. Cl. CPC A47L 11/4011; A47L 11/4002; A47L 11/4061; A61L 2/10; A61L 2/24; B25J 9/0009; G05D 1/435; G05D 1/648; A47L 2201/04; A47L 2201/06; A61L 2202/11; A61L 2202/14; A61L 2202/16; A61L 2202/17; G05D 2109/12; G06V 10/82; G06V 20/58

(57) ABSTRACT

The present invention discloses a system and a method for autonomous artificial intelligence-based (AI-based) cleaning of diverse surfaces. The system obtains sensor data. The system determines spatial characteristics of the diverse surfaces. The system changes a height of an autonomous cleaning device. The system generates a road map to navigate the autonomous cleaning device to a targeted surface. The system determines geometrical features and contamination data on the targeted surface. The system determines a corresponding cleaning equipment and a quantity of a cleaning agent. The system maintains a consistent contact between the targeted surface and the corresponding cleaning equipment. The system dispenses the determined quantity of the cleaning agent on to the corresponding cleaning equipment. The system initiates a first-round of cleaning. The system identifies particulate accumulation and contaminants on the targeted surface to initiate a second-round cleaning.

### EARLIEST PRIORITY DATE

This application claims priority from a Provisional patent application filed in India having patent application No. 202541038921, filed on 23rd April 2025 and titled “SYSTEM AND METHOD FOR AUTONOMOUS ARTIFICIAL INTELLIGENCE-BASED (AI-BASED) CLEANING OF DIVERSE SURFACES”.

### FIELD OF INVENTION

Embodiments of the present invention relate to intelligent and adaptive autonomous cleaning systems, specifically a system and a method for autonomous artificial intelligence-based (AI-based) cleaning of diverse surfaces, including but not limited to industrial, commercial, and residential environments.

### BACKGROUND

Sanitation and hygiene have always been critical aspects of public health and infrastructure maintenance. Cleaning large public spaces such as railway stations, airports, malls, and commercial buildings requires significant manual labour and resources. Traditional cleaning methods involve human-operated equipment and manual labour, which may not always ensure thorough sanitation, especially in areas with high footfall and surfaces of varying heights and contours. Over the years, cleaning technologies have evolved from simple mopping techniques to sophisticated mechanized cleaning equipment, but most of the cleaning methods still require human intervention to adjust height, detect contamination levels, and control cleaning force dynamically.

Automated cleaning robots have gained popularity in recent years, particularly for domestic floor cleaning applications. Robotic vacuum cleaners have been integrated with basic artificial intelligence (AI) and sensor-based navigation to autonomously clean residential spaces. However, the automated cleaning robots and the robotic vacuum cleaners are primarily designed for smooth surfaces and relatively small, enclosed environments. In industrial and commercial settings, larger-scale cleaning robots have also been deployed, but the larger-scale cleaning robots are manually controlled and operate with limited automation. The larger-scale cleaning robots lack advanced decision-making capabilities, making the larger-scale cleaning robots unsuitable for complex cleaning tasks that require real-time contamination detection, dynamic force control, and AI-driven navigation in cluttered environments. The larger-scale cleaning robots struggle with multi-surface cleaning, as they lack height adaptation mechanism and cannot detect surface irregularities effectively.

In the domain of public restrooms, maintaining hygiene is particularly challenging due to high usage and the presence of various contaminants. Some semi-automated toilet cleaning systems exist, but the semi-automated toilet cleaning systems are restricted to specific toilet structures and do not adjust cleaning intensity based on contamination levels. Furthermore, existing robotic solutions for cleaning the public restrooms and large areas lack the ability to assess cleanliness scores and instead rely on predefined routines, which are ineffective in handling real-time cleaning requirements.

Existing technologies disclose smart trolleys and self-cleaning robots. However, the smart trolleys and the self-cleaning robots have limitations in cleaning different types of toilet structures and floors in large real estate spaces. Some robotic cleaning solutions exist in the market, but the robotic cleaning solutions lack AI capabilities for adaptive force application during cleaning. Some robotic cleaning solutions employ blue-ray-based cleaning but may not incorporate AI-driven navigation and physical scrubbing mechanisms. The robotic cleaning solutions fail to perform multiple rounds of cleaning based on surface conditions, and do not integrate cloud-based cleaning analytics.

Most existing robotic cleaning solutions focus on predefined cleaning paths, manual operation, and limited automation that may not consider real-time adjustments based on surface conditions. Additionally, large platform cleaning solutions generally require human supervision to drive and control the machines, making the large platform cleaning solutions inefficient for large-scale public cleaning applications.

Therefore, there is a need for a fully autonomous cleaning system that integrates advanced AI with robotic mechanisms to effectively clean large commercial spaces and restroom facilities. Such a system should be capable of adapting to various cleaning conditions, identifying areas that require more intensive cleaning, and operating without the human intervention to ensure consistent hygiene standards.

### SUMMARY

This summary is provided to introduce a selection of concepts, in a simple manner, which is further described in the detailed description of the disclosure. This summary is neither intended to identify key or essential inventive concepts of the subject matter nor to determine the scope of the disclosure.

In order to overcome the above deficiencies of the prior art, the present disclosure is to solve the technical problem by providing a system for autonomous artificial intelligence-based (AI-based) cleaning of diverse surfaces.

In accordance with an embodiment of the present invention, the system for the autonomous AI-based cleaning of the diverse surfaces is disclosed. The system comprises an autonomous cleaning device. The autonomous cleaning device comprises a plurality of articulated shafts and a fixture. The fixture comprises a plurality of sensors, a rotating arm, an angle arm assembly, a microcontroller unit, and a memory unit.

Yet in another embodiment, the plurality of articulated shafts comprises a proximal end and a distal end. The plurality of articulated shafts is configured to change a height of the autonomous cleaning device based on the diverse surfaces. The proximal end is operatively connected to one or more support frames. The distal end of each articulated shaft of the plurality of articulated shafts is connected to a wheel assembly. The plurality of articulated shafts is selected from a group that comprises at least one of: telescopic shafts, hydraulic shafts, pneumatic shafts, lead screw-driven shafts, rack and pinion shafts, scissor lift shafts, ball screw shafts, and electromechanical actuator shafts.

Yet in another embodiment, the fixture comprises a first surface and a second surface. The fixture is operatively connected to the one or more support frames. Yet in another embodiment, the plurality of sensors is operatively positioned on at least one of: the first surface and the second surface. The plurality of sensors is configured to generate sensor data comprises at least one of: depth perception data, bacterial contamination data, inertial measurement data, and chemical compositions. The plurality of sensors comprises at least one of: an obstacle detection unit, an inertial measurement unit (IMU), at least one of: one or more infrared (IR) sensors, one or more ultraviolet (UV) sensors, one or more ultrasonic sensors, and one or more chemical sensors.

The obstacle detection unit is configured to generate the depth perception data by computing a distance between the autonomous cleaning device and one or more obstacles for detecting the one or more obstacles on the diverse surfaces. The IMU is configured to determine acceleration and orientation of the autonomous cleaning device for generating the inertial measurement data. At least one of: the one or more IR sensors and one or more ultraviolet (UV) sensors are configured to generate the bacterial contamination data based on determining bacterial contamination levels on the targeted surface before and after cleaning. The one or more ultrasonic sensors are configured to generate the one or more geometrical features of the targeted surface. The one or more chemical sensors are configured to detect the chemical compositions on the targeted surface for dispensing an optimised quantity of a cleaning agent.

Yet in another embodiment, the rotating arm protrudes from the second surface. The rotating arm is connected to a first actuation unit that is operatively positioned on the first surface. The rotating arm is configured with a holder to hold a visual data capturing unit at a defined angle. The rotating arm is configured to rotate the visual data capturing unit in a defined direction at a time of capturing first visual data of the diverse surfaces.

Yet in another embodiment, the angle arm assembly is pivotally connected to a free end of the rotating arm through a second actuation unit. The angle arm assembly is configured to change a position of at least one of: a liquid tank and a cleaning equipment assembly at the defined angle to reach the diverse surfaces. The cleaning equipment assembly comprises at least one of: a cleaning equipment holder, a cleaning equipment actuation unit, and one or more cleaning equipment. The cleaning equipment holder is operatively engaged with a fifth actuation unit. The cleaning equipment holder is configured to hold the cleaning equipment actuation unit. The one or more cleaning equipment are operatively connected to the cleaning equipment actuation unit for cleaning in the pre-defined sequential patterns. The pre-defined sequential patterns comprise at least one of: a zig-zag pattern, a circular pattern, a radial pattern, a spiral pattern, a grid pattern, and a randomized adaptive pattern. The one or more cleaning equipment comprises at least one of: a soft bristle brush, a hard bristle brush, an antimicrobial brush, a microfiber cleaning pad, a rotating sponge head, a silicone scraper, an electrostatic dusting pad, and an ultraviolet (UV) sterilization module.

Yet in another embodiment, the autonomous cleaning device is configured with a radio frequency (RF) unit. The RF unit is configured to: a) connect the autonomous cleaning device to one or more servers for generating the cleaning statistics and b) integrate with Internet of Things (IoT)-based facility management systems for automated scheduling of the autonomous cleaning device and reporting to the one or more users.

Yet in another embodiment, the microcontroller unit is operatively positioned on the first surface. Yet in another embodiment, the memory unit is operatively connected to the microcontroller unit. The memory unit comprises a set of computer-readable instructions in form of a plurality of subsystems, configured to be executed by the microcontroller unit. The plurality of subsystems comprises a data obtaining subsystem, a spatial recognition subsystem, an adaptative height alteration subsystem, a navigation subsystem, a surface scanning subsystem, a decontamination subsystem, an intelligent spot detection subsystem, and a notification subsystem.

Yet in another embodiment, the data obtaining subsystem is configured to obtain the sensor data for initiating the cleaning of the diverse surfaces. Yet in another embodiment, the spatial recognition subsystem is configured to trigger the first actuation unit to actuate the rotating arm to capture the first visual data of the diverse surfaces for determining one or more spatial characteristics of the diverse surfaces. The one or more spatial characteristics comprise at least one of: coordinates of the targeted surface, height of the targeted surface, confined spaces, edges, and corners on the diverse surfaces, surface material type, and surface texture.

Yet in another embodiment, the adaptative height alteration subsystem is configured to trigger a third actuation unit and a fourth actuation unit associated with the plurality of articulated shafts and the wheel assembly respectively to at least one of: a) change the height of the autonomous cleaning device based on the one or more spatial characteristics and b) regulate speed of the fourth actuation unit to move the autonomous cleaning device based on the one or more spatial characteristics.

Yet in another embodiment, the navigation subsystem is configured to: a) generate obstacle avoidance data by analysing at least one of: the first visual data, the depth perception data, and the IR data through at least one of: one or more predictive motion models, one or more deep reinforcement learning models and b) generate a road map to navigate the autonomous cleaning device to a targeted surface within the diverse surfaces based on processing the obstacle avoidance data and the first visual data through at least one of: one or more path planning models, one or more reinforcement learning models, and one or more motion planning models. At least one of: the one or more predictive motion models, the one or more deep reinforcement learning models are trained on at least one of: historical movement data, obstacle interaction scenarios, Simulated robotic movement datasets, Reinforcement learning-based training simulations, Simultaneous Localization and Mapping (SLAM) datasets. At least one of: the one or more path planning models, the one or more reinforcement learning models, and the one or more motion planning models are trained on dynamic navigation datasets. The dynamic navigation datasets comprise at least one of: a) terrain recognition data for altering motion parameters based on the one or more spatial characteristics and b) obstacle-rich real-world scenario data for adaptive movement decision-making.

Yet in another embodiment, the surface scanning subsystem is configured with one or more convolutional neural networks (CNNs) to analyse the first visual data and the sensor data for determining at least one of: one or more geometrical features and contamination data on the targeted surface. The one or more geometrical features of the targeted surface comprise at least one of: surface curvature profiles, edge contours, depth variations, surface area dimensions, inclination angles, surface irregularities, and symmetry and shape classification. The contamination data comprises at least one of: the contaminants, surface stain intensity, bacterial presence data, chemical residue detection, organic waste detection, liquid spill analysis, dust and the particulate accumulation, and Odor intensity data. The one or more CNNs are trained on image-based contamination datasets, wherein training includes at least one of: a) labelled contamination datasets for classifying the contamination data and the one or more geometrical features, b) supervised learning datasets for analysing the contamination data to predict a cleaning intensity, and c) labelled geometric feature datasets for analysing the one or more geometrical features to recognize at least one of: concave surfaces, convex surfaces, irregular surfaces, boundaries, edges, corners, and surface depth variations.

Yet in another embodiment, the decontamination subsystem is configured to: a) determine at least one of: a corresponding cleaning equipment from the one or more cleaning equipment and a quantity of a cleaning agent, based on analysing the contamination data through the one or more CNNs, b) maintain a consistent contact between the targeted surface and the corresponding cleaning equipment by triggering the fifth actuation unit and a sixth actuation unit associated with the cleaning equipment assembly and the liquid tank respectively by analysing the one or more geometrical features through a gyroscopic stabilization mechanism, c) trigger a seventh actuation unit associated with the liquid tank to dispense the determined quantity of the cleaning agent on to the corresponding cleaning equipment based on the contamination data, and d) trigger the cleaning equipment actuation unit for initiating a first-round of cleaning in pre-defined sequential patterns on the targeted surface based on the contamination data.

Yet in another embodiment, the intelligent spot detection subsystem is configured to activate at least one of: the visual data capturing unit and the one or more chemical sensors to capture second visual data to identify at least one of: particulate accumulation and contaminants on the targeted surface to initiate a second-round cleaning. The intelligent spot detection subsystem is configured to compare the first visual data with the second visual data to quantitatively assess a cleanliness score and adapt the cleaning intensity accordingly. The cleanliness score is determined based on processing the first visual data and the second visual data through at least one of: difference mapping models and threshold-based scoring procedures.

Yet in another embodiment, the notification subsystem is configured to provide one or more alert notifications to one or more users regarding at least one of: cleaning statistics, cleaning agent refilling status, power level status, system performance diagnostics, sensor calibration alerts, real-time operational status, and model update alerts.

In accordance with an embodiment of the present invention, a method for the autonomous AI-based cleaning of the diverse surfaces is disclosed. In the first step, the method includes generating, by the plurality of sensors, the sensor data that comprises at least one of: the depth perception data, the bacterial contamination data, the inertial measurement data, and the chemical compositions.

In the next step, the method includes obtaining, by the microcontroller unit through the data obtaining subsystem, the sensor data for initiating the cleaning of diverse surfaces.

In the next step, the method includes triggering, by the microcontroller unit through the spatial recognition subsystem, the first actuation unit associated with the rotating arm configured with the visual data capturing unit to capture the first visual data of the diverse surfaces to determine the one or more spatial characteristics of the diverse surfaces.

In the next step, the method includes changing, by the microcontroller unit through the adaptative height alteration subsystem, the height of the autonomous cleaning device based on the one or more spatial characteristics through the third actuation unit associated with the plurality of articulated shafts.

In the next step, the method includes regulating, by the microcontroller unit through the adaptative height alteration subsystem, the speed of the fourth actuation unit associated with the wheel assembly to move the autonomous cleaning device based on the one or more spatial characteristics.

In the next step, the method includes analysing, by the microcontroller unit through the navigation subsystem, at least one of: the first visual data, the depth perception data, and the IR data through at least one of: the one or more predictive motion models, the one or more deep reinforcement learning models to generate the obstacle avoidance data.

In the next step, the method includes processing, by the microcontroller unit through the navigation subsystem, the obstacle avoidance data and the first visual data through at least one of: the one or more path planning models, the one or more reinforcement learning models, and the one or more motion planning models to generate the road map to navigate the autonomous cleaning device to the targeted surface within the diverse surfaces.

In the next step, the method includes analysing, by the microcontroller unit through the surface scanning subsystem configured with the one or more CNNs, the first visual data and the sensor data for determining at least one of: the one or more geometrical features and the contamination data on the targeted surface.

In the next step, the method includes analysing, by the microcontroller unit through the decontamination subsystem, the contamination data through the one or more CNNs to determine at least one of: the corresponding cleaning equipment from the one or more cleaning equipment and the quantity of the cleaning agent.

In the next step, the method includes analysing, by the microcontroller unit through the decontamination subsystem, the one or more geometrical features through the gyroscopic stabilization mechanism to maintain the consistent contact between the targeted surface and the corresponding cleaning equipment through the fifth actuation unit and the sixth actuation unit associated with the cleaning equipment assembly and the liquid tank respectively.

In the next step, the method includes triggering, by the microcontroller unit through the decontamination subsystem, the seventh actuation unit associated with the liquid tank to dispense the determined quantity of the cleaning agent on to the corresponding cleaning equipment based on the contamination data.

In the next step, the method includes triggering, by the microcontroller unit through the decontamination subsystem, the cleaning equipment actuation unit to initiate the first-round of cleaning in the pre-defined sequential patterns on the targeted surface based on the contamination data.

In the next step, the method includes activating, by the microcontroller unit through the intelligent spot detection subsystem, at least one of: the visual data capturing unit the one or more chemical sensors to capture the second visual data to identify at least one of: the particulate accumulation and the contaminants on the targeted surface to initiate the second-round cleaning.

To further clarify the advantages and features of the present invention, a more particular description of the invention will follow by reference to specific embodiments thereof, which are illustrated in the appended figures. It is to be appreciated that these figures depict only typical embodiments of the invention and are therefore not to be considered limiting in scope. The invention will be described and explained with additional specificity and detail with the appended figures.

### BRIEF DESCRIPTION OF THE DRAWINGS

The disclosure will be described and explained with additional specificity and detail with the accompanying figures in which:

FIG. 1A illustrates an exemplary block diagram of an autonomous cleaning device, in accordance with an embodiment of the present invention;

FIG. 1B illustrates an exemplary isometric view of the autonomous cleaning device, in accordance with an embodiment of the present invention;

FIG. 1C illustrates an exemplary exploded view of the autonomous cleaning device, in accordance with an embodiment of the present invention;

FIG. 1D illustrates an exemplary isometric view of an angle arm assembly associated with the autonomous cleaning device, in accordance with an embodiment of the present invention;

FIG. 1E illustrates an exemplary isometric view of a liquid tank assembly associated with the autonomous cleaning device, in accordance with an embodiment of the present invention;

FIG. 1F illustrates an exemplary side view of the autonomous cleaning device, in accordance with an embodiment of the present invention;

FIG. 1G illustrates an exemplary front view of the autonomous cleaning device, in accordance with an embodiment of the present invention;

FIGS. 1H-1K illustrate exemplary visual representations depicting an optimal placement of the autonomous cleaning device on diverse surfaces, in accordance with an embodiment of the present invention;

FIG. 2 illustrates an exemplary block diagram representation of a network architecture depicting a system for autonomous artificial intelligence-based (AI-based) cleaning of the diverse surfaces, in accordance with an embodiment of the present invention;

FIG. 3 illustrates an exemplary block diagram representation of the system as shown in FIG. 1 for the autonomous AI-based cleaning of the diverse surfaces, in accordance with an embodiment of the present invention; and

FIG. 4 illustrates an exemplary flow diagram representation depicting a method for the autonomous AI-based cleaning of the diverse surfaces, in accordance with an embodiment of the present invention.

Further, those skilled in the art will appreciate that elements in the figures are illustrated for simplicity and may not have necessarily been drawn to scale. Furthermore, in terms of the method steps, chemical compounds, equipment and parameters used herein may have been represented in the figures by conventional symbols, and the figures may show only those specific details that are pertinent to understanding the embodiments of the present disclosure so as not to obscure the figures with details that will be readily apparent to those skilled in the art having the benefit of the description herein.

### DETAILED DESCRIPTION OF THE PRESENT INVENTION

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated system, and such further applications of the principles of the disclosure as would normally occur to those skilled in the art are to be construed as being within the scope of the present disclosure.

The terms “comprises”, “comprising”, or any other variations thereof, are intended to cover a non-exclusive inclusion, such that a process or method that comprises a list of steps does not include only those steps but may include other steps not expressly listed or inherent to such a process or method. Similarly, one or more components, compounds, and ingredients preceded by “comprises . . . a” does not, without more constraints, preclude the existence of other components or compounds or ingredients or additional components. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this disclosure belongs. The system, methods, and examples provided herein are only illustrative and not intended to be limiting.

In the following specification and the claims, reference will be made to a number of terms, which shall be defined to have the following meanings. The singular forms “a”, “an”, and “the” include plural references unless the context clearly dictates otherwise.

A computer system (standalone, client or server computer system) configured by an application may constitute a “module” (or “subsystem”) that is configured and operated to perform certain operations. In one embodiment, the “module” or “subsystem” may be implemented mechanically or electronically, so a module include dedicated circuitry or logic that is permanently configured (within a special-purpose processor) to perform certain operations. In another embodiment, a “module” or “subsystem” may also comprise programmable logic or circuitry (as encompassed within a general-purpose processor or other programmable processor) that is temporarily configured by software to perform certain operations.

Accordingly, the term “module” or “subsystem” should be understood to encompass a tangible entity, be that an entity that is physically constructed permanently configured (hardwired) or temporarily configured (programmed) to operate in a certain manner and/or to perform certain operations described herein.

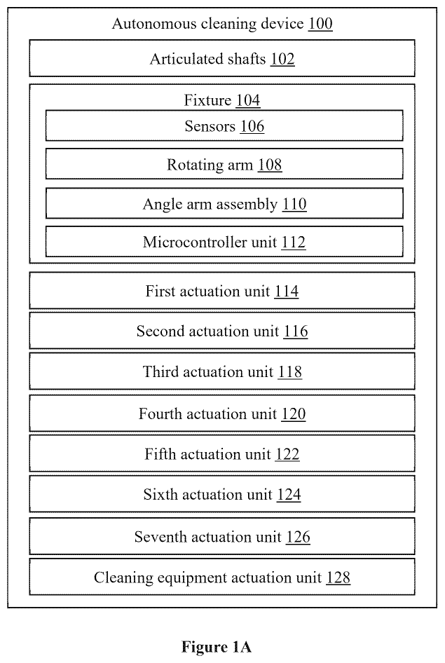

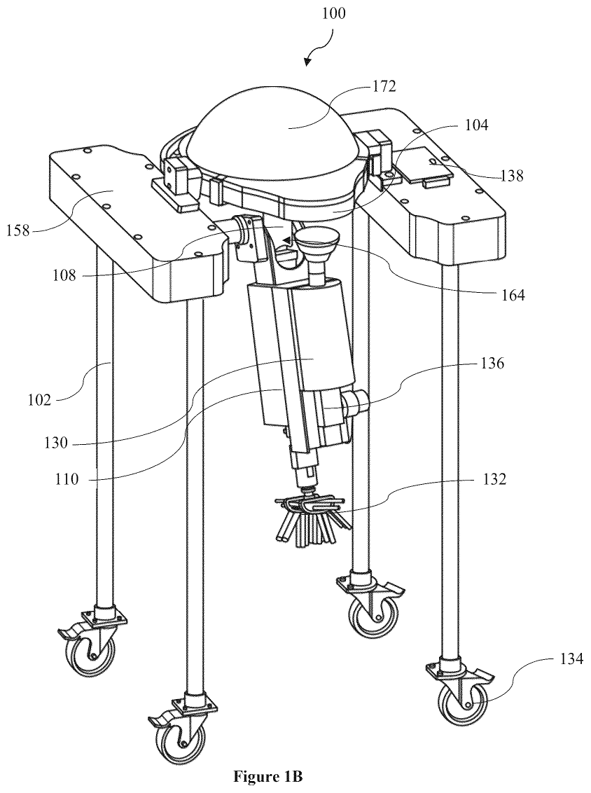

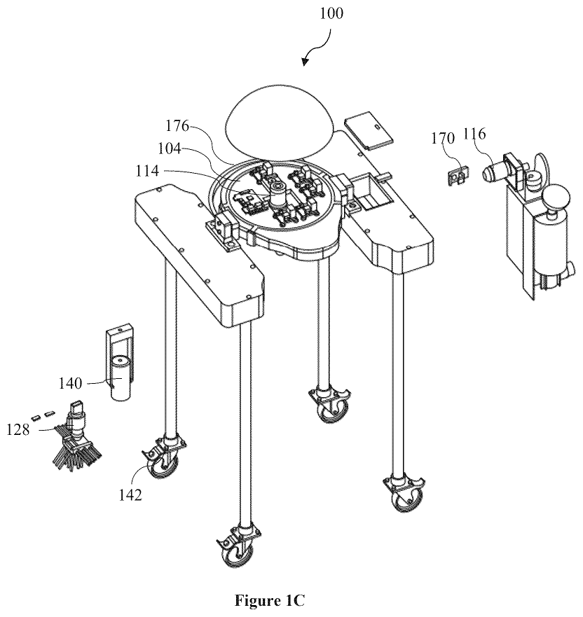

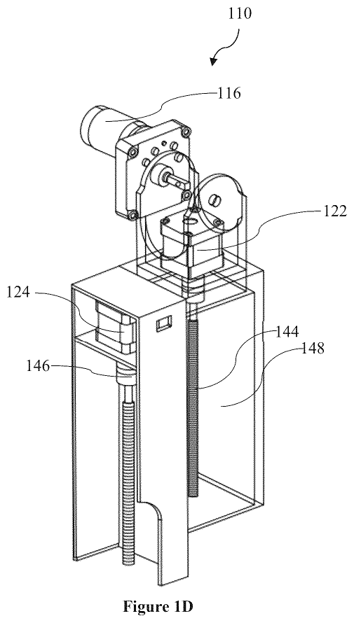

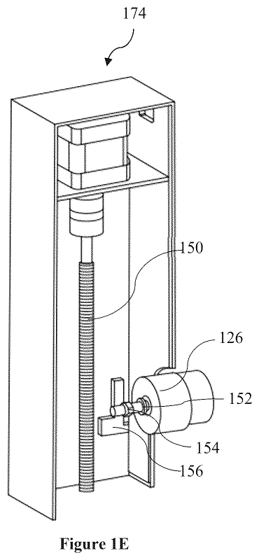

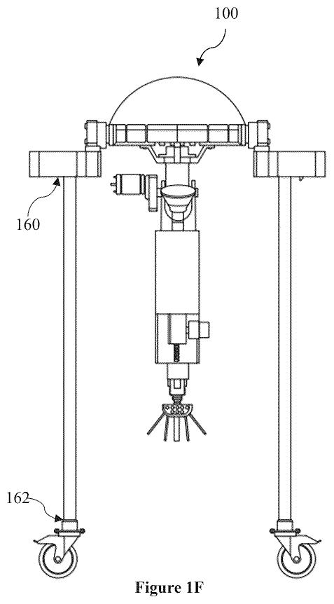

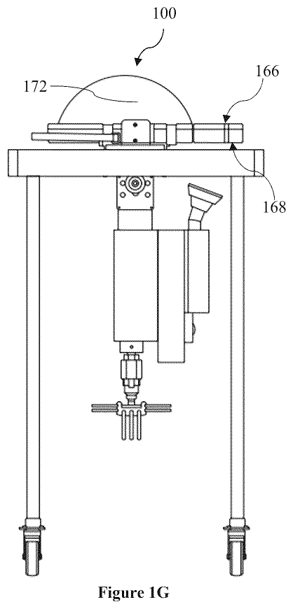

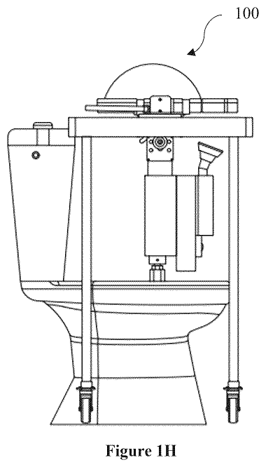

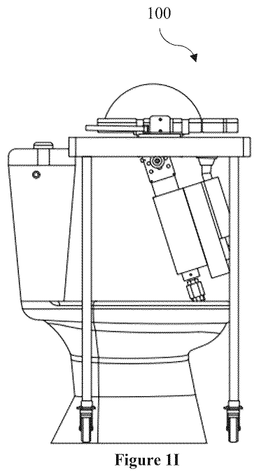

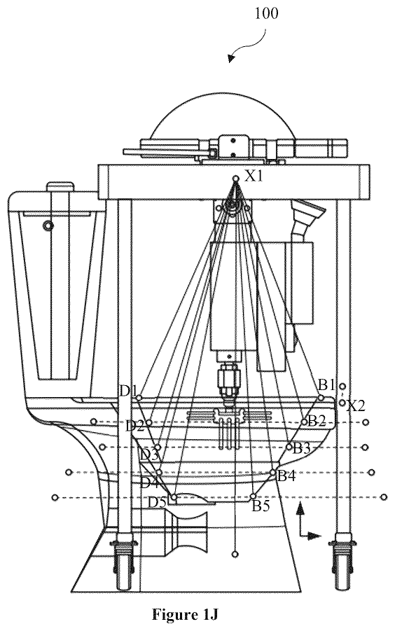

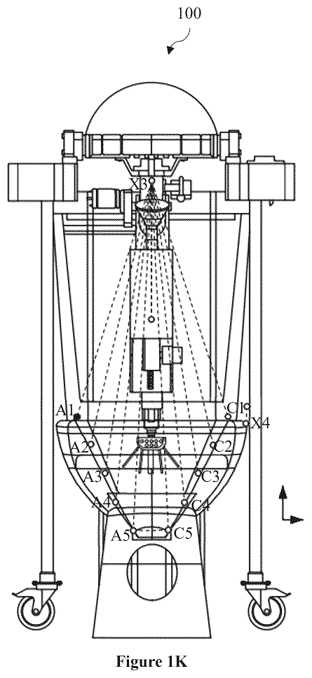

Referring now to the drawings, and more particularly to FIG. 1A through FIG. 4, where similar reference characters denote corresponding features consistently throughout the figures, there are shown preferred embodiments, and these embodiments are described in the context of the following exemplary system and/or method.

Embodiments of the present invention relate to a system for autonomous artificial intelligence-based (AI-based) cleaning of diverse surfaces.

FIG. 1A illustrates an exemplary block diagram of an autonomous cleaning device 100, in accordance with an embodiment of the present invention;

FIG. 1B illustrates an exemplary isometric view of the autonomous cleaning device 100, in accordance with an embodiment of the present invention;

FIG. 1C illustrates an exemplary exploded view of the autonomous cleaning device 100, in accordance with an embodiment of the present invention;

FIG. 1D illustrates an exemplary isometric view of an angle arm assembly 110 associated with the autonomous cleaning device 100, in accordance with an embodiment of the present invention;

FIG. 1E illustrates an exemplary isometric view of a liquid tank assembly 174 associated with the autonomous cleaning device 100, in accordance with an embodiment of the present invention;

FIG. 1F illustrates an exemplary side view of the autonomous cleaning device 100, in accordance with an embodiment of the present invention;

FIG. 1G illustrates an exemplary front view of the autonomous cleaning device 100, in accordance with an embodiment of the present invention; and

FIGS. 1H-1K illustrate exemplary visual representations depicting an optimal placement of the autonomous cleaning device 100 on diverse surfaces, in accordance with an embodiment of the present invention.

According to an exemplary embodiment of the disclosure, the system for the autonomous AI-based cleaning of the diverse surfaces is disclosed. The system comprises the autonomous cleaning device 100. The autonomous cleaning device 100 comprises a plurality of articulated shafts 102 and a fixture 104. The fixture 104 comprises a plurality of sensors 106, a rotating arm 108, the angle arm assembly 110, a microcontroller unit 112, a memory unit, and a plurality of drivers (not shown) associated with a plurality of actuation units. The diverse surfaces comprise, but not limited to, at least one of: commodes, floors, urinals, sinks, bathtubs, countertops, mirrors, glass panels, walls, tiles, carpets, rugs, wooden surfaces, stainless steel surfaces, escalator steps, handrails, door handles, elevator buttons, kitchen countertops, dining tables, workstations, hospital beds, medical equipment surfaces, gym equipment, high-touch surfaces in public areas, and the like.

In an exemplary embodiment, each articulated shaft 102 of the plurality of articulated shafts 102 is configured with a proximal end 160 operatively connected to one or more support frames 158 and a distal end 162 linked to a wheel assembly. The proximal end 160 is an upper end of each articulated shaft 102. The distal end 162 is a lower end of each articulated shaft 102. The wheel assembly comprises a shaft stand 142 and a wheel 134. The shaft stand 142 is operatively connected to each articulated shaft 102. The shaft stand 142 is configured to stabilize the autonomous cleaning device 100 at least one of: in motion and during cleaning operations. The wheel 134 is operatively connected to the shaft stand 142. The wheel 134 is configured to provide a smooth and controlled movement across the diverse surfaces. The wheel assembly is configured to support at least one of: a manual movement and an autonomous movement of the autonomous cleaning device 100.

The plurality of articulated shafts 102 enables height adjustment based on the diverse surfaces, allowing the autonomous cleaning device 100 to maintain stability and optimal positioning during the cleaning operations. The plurality of articulated shafts 102 facilitates smooth movement across irregular and sloped surfaces associated with the diverse surfaces, ensuring uninterrupted functionality. This height-adjustment mechanism enhances adaptability, making the autonomous cleaning device 100 suitable for a wide range of cleaning applications where surface variations are common.

The plurality of articulated shafts 102 is configured to hold the one or more support frames 158. The plurality of articulated shafts 102 supports any sort of shocks while the autonomous cleaning device 100 is in a momentum state. The one or more support frames 158 are configured to provide a structural integrity to each articulated shaft 102 for providing a controlled movement to the autonomous cleaning device 100 across the diverse surfaces.

The plurality of articulated shafts 102 is selected from a group that comprises, but not restricted to, at least one of: telescopic shafts, hydraulic shafts, pneumatic shafts, lead screw-driven shafts, rack and pinion shafts, scissor lift shafts, ball screw shafts, electromechanical actuator shafts, and the like. The telescopic shafts extend and retract linearly to modify the height dynamically. The hydraulic shafts employ a pressurised fluid for a controlled vertical displacement, while the pneumatic shafts rely on compressed air to achieve similar functionality. The lead screw-driven shafts convert a rotational motion into a precise linear displacement, whereas the rack and pinion shafts employ interlocking gears for smooth height changes. The scissor lift shafts employ an intersecting framework to provide stable elevation adjustments. The ball screw shafts integrate ball bearings to ensure efficient and precise linear motion. The electromechanical actuator shafts employ motorized mechanisms for the controlled vertical displacement. The plurality of articulated shafts 102 allows the autonomous cleaning device 100 to adapt the height effectively, ensuring enhanced mobility and stability across varying diverse surfaces. For instance, in a floor wiping mode, the height of the plurality of articulated shafts 102 is adjusted to reach a floor level associated with the diverse surfaces.

In an exemplary embodiment, the fixture 104 comprises a first surface 166 and a second surface 168. The first surface 166 is an upper surface of the fixture 104 and the second surface 168 is a lower surface of the fixture 104. The fixture 104 is operatively connected to the one or more support frames 158, providing structural stability to the autonomous cleaning device 100. The fixture 104 is a mounting base for various functional components, ensuring precise positioning and effective operation. By providing the first surface 166 and the second surface 168, the fixture 104 accommodates multiple attachments, enhancing the adaptability of the autonomous cleaning device 100 to different cleaning environments. The integration of the fixture 104 with the one or more support frames 158 contributes to the structural integrity of the autonomous cleaning device 100, enabling efficient movement and cleaning performance.

In another exemplary embodiment, an enclosure 172 is operatively positioned on the fixture 104 to enclose and protect the functional components from at least one of: dust, water, and other contaminants. The automated cleaning device 100 is also configured with a holding ring 176. The holding ring 176 is operationally positioned around the enclosure 172. The holding ring 176 allows one or more users to carry the automated cleaning device 100 with ease.

In an exemplary embodiment, the plurality of sensors 106 is operatively positioned on at least one of: the first surface 166 and the second surface 168 of the fixture 104. The plurality of sensors 106 is configured to generate the sensor data. The sensor data may comprise, but not constricted to, at least one of: depth perception data, bacterial contamination data, inertial measurement data, chemical compositions, and the like. The plurality of sensors 106 may comprise, but not constrained to, at least one of: an obstacle detection unit, an inertial measurement unit (IMU), at least one of: one or more infrared (IR) sensors and one or more ultraviolet (UV) sensors, one or more ultrasonic sensors, one or more chemical sensors, and the like.

In an exemplary embodiment, the obstacle detection unit is configured to generate the depth perception data by computing a distance between the autonomous cleaning device 100 and one or more obstacles present on the diverse surfaces. This enables real-time obstacle detection, ensuring efficient navigation and collision avoidance. The IMU is configured to determine an acceleration and an orientation of the autonomous cleaning device 100, thereby generating the inertial measurement data that aids in stabilising the movement across the diverse surfaces. At least one of: the one or more IR sensors and the one or more UV sensors assess bacterial contamination levels on a targeted surface before and after cleaning, thereby enabling data-driven sanitation verification. At least one of: the one or more IR sensors and the one or more UV sensors are configured to generate the bacterial contamination data based on the determined bacterial contamination levels. The one or more ultrasonic sensors analyse one or more geometrical features of the targeted surface on the diverse surfaces, ensuring optimal cleaning coverage. Additionally, the one or more chemical sensors detect surface chemical compositions, allowing the autonomous cleaning device 100 to dispense an optimized quantity of a cleaning agent based on detected contaminants on the diverse surfaces. By integrating the plurality of sensors 106, the autonomous cleaning device 100 ensures superior cleaning performance with enhanced efficiency and resource management.

In an alternative embodiment, the plurality of sensors 106 may include one or more depth sensors configured to detect surface contours and measure the distance between one or more cleaning equipment 132 and the diverse surfaces for optimal navigation and cleaning of the diverse surfaces, and one or more Light Detection and Ranging (LiDAR) sensors configured to create high-resolution three-dimensional (3D) maps of the diverse surfaces for accurate positioning and obstacle detection.

In another exemplary embodiment, the plurality of sensors 106 may comprise, but not limited to, at least one of: one or more speed sensors, one or more position sensors, one or more temperature sensors, and the like. The one or more speed sensors are operatively connected to the plurality of actuation units. The one or more speed sensors are configured to measure a rotational speed of the plurality of actuation units for optimal control of the one or more cleaning equipment 132. The one or more position sensors are operatively connected to the plurality of actuation units. The one or more position sensors are configured to track an angular position of a plurality of shafts of the plurality of actuation units for accurate positioning of at least one of: the angle arm assembly 110 and a liquid tank 130 associated with the liquid tank assembly 174 during the cleaning operations. The one or more temperature sensors are operatively connected to the plurality of actuation units. The one or more temperature sensors are configured to monitor an operating temperature of the plurality of actuation units to prevent overheating.

In an exemplary embodiment, the rotating arm 108 protrudes from the second surface 168. The rotating arm 108 is operatively connected to a first actuation unit 114 associated with the plurality of actuation units positioned on the first surface 166. The rotating arm 108 is configured to support a visual data capturing unit (e.g., a camera) through an integrated holder 170, ensuring stable positioning at a defined angle. This configuration allows precise alignment of the visual data capturing unit to enhance imaging accuracy for monitoring and analysis. The rotating arm 108 enables controlled motion of the visual data capturing unit, allowing the visual data capturing unit to rotate in a defined direction while capturing first visual data of the diverse surfaces. The defined direction is in at least one of: a rotating 360-degree arc and a predefined angular range, enabling comprehensive scanning of the diverse surfaces while dynamically adjusting a field of view based on one or more spatial characteristics and cleaning requirements. This controlled movement ensures comprehensive scanning of the targeted surface, facilitating real-time assessment of surface conditions. By leveraging this mechanism, the autonomous cleaning device 100 enhances the capability to detect the contaminants, monitor cleaning efficiency, and optimize cleaning paths based on the captured first visual data. In an illustrative embodiment, the first actuation unit 114 is a direct current (DC) motor. The first actuation unit 114 is configured to perform a 360-degree rotation of the entire angle arm assembly 110 via the rotating arm 108.

The visual data capturing unit is configured to, but not constrained to, at least one of: a) detect the targeted surface and a size of the targeted surface, b) identify the size and shape of the one or more obstacles, c) identify a virus associated with the contaminants on the targeted surface, and d) capture the first visual data and process the level of the cleaning agent.

In an exemplary embodiment, the angle arm assembly 110 is pivotally connected to a free end 164 of the rotating arm 108 through a second actuation unit 116 associated with the plurality of actuation units, thereby allowing controlled movement for effective positioning of at least one of: the liquid tank 130 and a cleaning equipment assembly. The second actuation unit 116 enables at least one of: the liquid tank 130 and the cleaning equipment assembly to be adjusted at the defined angle, ensuring optimal reach to the diverse surfaces. In an illustrative embodiment, the second actuation unit 116 is a direct current (DC) motor (e.g., a square gearbox DC motor). The second actuation unit 116 is configured to alter an angle of the angle arm assembly 110, which depends on the targeted surface. The second actuation unit 116 is controlled using the microcontroller unit 112.

The cleaning equipment assembly may comprise, but not constricted to, at least one of: a cleaning equipment holder 140, a cleaning equipment actuation unit 128 (motor) associated with the plurality of actuation units, the one or more cleaning equipment 132, and the like. The cleaning equipment holder 140 is operatively engaged with a fifth actuation unit 122 associated with the plurality of actuation units. The cleaning equipment holder 140 serves as a mounting platform for securely holding the cleaning equipment actuation unit 128, thereby facilitating the efficient operation of the one or more cleaning equipment 132. The cleaning equipment holder 140 is configured to facilitate at least one of: quick insertion and removal of the one or more cleaning equipment 132.

The one or more cleaning equipment 132 are operatively connected to the cleaning equipment actuation unit 128. The cleaning equipment actuation unit 128 is configured to manoeuvre the one or more cleaning equipment 132 in pre-defined sequential patterns, ensuring comprehensive cleaning coverage. The pre-defined sequence patterns may include, but not restricted to, at least one of: a zig-zag pattern (linear movement with sharp directional changes for maximizing surface coverage), a circular pattern (rotational cleaning for even distribution of cleaning force), a radial pattern (expanding and contracting motions for systematic area coverage), a spiral pattern (continuous outward and inward movement for controlled scrubbing), a grid pattern (structured sweeping movement for uniform cleaning), randomized adaptive pattern (dynamic path adjustments based on real-time obstacle detection), and the like. The pre-defined sequential patterns optimize cleaning efficiency by adapting to different surface geometries and contamination levels.

The autonomous cleaning device 100 may adapt cleaning patterns based on at least one of: the pre-defined sequential patterns, user-defined cleaning patterns, and the real-time identification of the contaminants on the diverse surfaces.

The one or more cleaning equipment 132 may comprise, but not constricted to, at least one of: a soft bristle brush (gentle cleaning for delicate diverse surfaces), a hard bristle brush (abrasive scrubbing for tough stains), an antimicrobial brush (integrated antimicrobial properties to inhibit bacterial growth), a microfiber cleaning pad (high-absorption pad for trapping dust and liquid residues), a rotating sponge head (motorized scrubbing for effective stain removal), a silicone scraper (flexible blade for removing adhered debris), an electrostatic dusting pad (charged pad for capturing fine dust particles), an UV sterilization module (UV light-based disinfection to eliminate microbial contaminants), and the like. By integrating the one or more cleaning equipment 132, the autonomous cleaning device 100 ensures thorough cleaning across the diverse surfaces, maintaining hygiene and efficiency.

The one or more cleaning equipment 132 may perform uniform scrubbing in all areas without missing any spot by dynamically adjusting the height of at least one of: the one or more cleaning equipment 132 and the plurality of articulated shafts 102. The autonomous cleaning device 100 is configured to adjust the angle of the one or more cleaning equipment 132 to reach every corner, considering the curvature of the diverse surfaces. The one or more cleaning equipment 132 are easy to remove and replace with new one or more cleaning equipment 132. The autonomous cleaning device 100 may be equipped with one of: a magnetic attachment system and a quick-lock attachment system for replacement of the one or more cleaning equipment 132. In another alternative embodiment, the autonomous cleaning device 100 may be integrated with at least one of: the UV sterilization module and a disinfectant spray for enhanced cleaning.

In an alternative embodiment, the autonomous cleaning device 100 is configured with an automated cleaning equipment replacement mechanism, allowing for seamless and autonomous switching of the one or more cleaning equipment 132 based on surface type, contamination level, and cleaning requirements. The autonomous cleaning device 100 comprises a cleaning equipment docking and a storage unit, operatively integrated within the autonomous cleaning device 100. The cleaning equipment docking and the storage unit includes multiple compartments, each configured to hold the one or more cleaning equipment 132. The autonomous cleaning device 100 is equipped with a robotic arm assembly and an actuated locking mechanism to facilitate automatic removal and replacement of the one or more cleaning equipment 132. The process operates as: a) the microcontroller unit 112 determines the corresponding cleaning equipment 132 based on the contamination data, the surface type, and the cleaning intensity requirements, b) the actuated locking mechanism, which includes an electromechanical release system (such as a servo-actuated clamp and a pneumatic locking pin), disengages the corresponding cleaning equipment 132 from the cleaning equipment holder 140, c) the robotic arm assembly, equipped with a gripper mechanism, retrieves the used cleaning equipment 132 and places the used cleaning equipment 132 back into a respective compartment within the cleaning equipment docking and the storage unit, d) the robotic arm selects the new cleaning equipment 132 from the storage unit, aligning the new cleaning equipment 132 with the cleaning equipment holder 140, and e) the actuated locking mechanism securely fastens the newly selected cleaning equipment 132 into place, ensuring stability and proper functionality during the cleaning operations.

In an exemplary embodiment, the autonomous cleaning device 100 is equipped with a radio frequency (RF) unit that enables wireless communication for real-time monitoring, scheduling, and reporting. The RF unit is configured to connect the autonomous cleaning device 100 to one or more servers to generate and store cleaning statistics. The RF unit is configured to integrate with Internet of Things (IoT)-based facility management systems to facilitate automated scheduling and reporting. This connectivity ensures seamless coordination between the autonomous cleaning device 100 and centralized management platforms, enabling optimized operation without manual intervention.

The cleaning statistics may include, but not limited to, at least one of: surface coverage efficiency, cleaning time, contamination level variations, power consumption, cleaning agent usage, and the like. The cleaning statistics may assist the one or more users in tracking performance and improving operational efficiency. Additionally, the integration with the IoT-based facility management systems allows the automated scheduling based on at least one of: predefined cleaning cycles, detected contamination levels, and facility-specific requirements. The RF unit also enables real-time reporting, sending one or more alert notifications and maintenance updates to the one or more users, ensuring continuous operation with minimal downtime. This smart connectivity enhances operational efficiency, reduces resource wastage, and ensures a data-driven approach to facility cleanliness management.

A Message Queuing Telemetry Transport (MQTT) protocol is configured to efficiently transmit data from the microcontroller unit 112 to the one or more servers (cloud), enabling lightweight and reliable communication for IoT Applications. The System Performs Analytics Using cloud services to provide actionable insights through dashboards on a user interface associated with one or more communication devices and the one or more alert notifications. The user interface enables real-time actuation unit monitoring and control.

In an illustrative embodiment, the microcontroller unit 112 is operatively positioned on the first surface 166. The microcontroller unit 112 operatively connects to the plurality of actuation units through a motor driver Hardware Attached on Top (HAT), which regulates Pulse Width Modulation (PWM) signals to control speed and direction. The microcontroller unit 112 is a Raspberry Pi 5 model 8 Giga Bytes (GB) (A compact, high-performance single-board computer). The Raspberry Pi features at least one of: a 2.4 Giga Hertz (GHz) quad-core, 64-bit advanced Reduced Instruction Set Computing (RISC) Machine (ARM) Cortex-A76 Central Processing Unit (CPU) with 512 Kilo Bytes Level 2 (L2) caches and a 2 Mega Bytes shared Level 3 (L3) cache for efficient processing, a VideoCore VII Graphics Processing Unit (GPU) for enhanced graphics, 8 GB Low Power Double Data Rate 4X (LPDDR4X-4267) Synchronous Dynamic Random Access Memory (SDRAM) for faster multitasking, dual-band 2.4 GHz and 5.0 GHz 802.11ac Wireless Fidelity (Wi-Fi) with Bluetooth® 5.0/Bluetooth Low Energy (BLE) for seamless connectivity; high-speed Secure Digital (SD) cards in SDR104 mode for storage, 2× Universal Serial Bus (USB) 3.0 ports (5 Gigabits per second (Gbps)) and 2× USB 2.0 ports for peripherals, Gigabit Ethernet (GbE) with Power over Ethernet Plus (PoE+) support (via PoE+HAT) for wired networking, and dual 4-lane Mobile Industry Processor Interface (MIPI) transceivers for high-speed camera/display integration.

In an exemplary embodiment, the one or more power supply units are operatively positioned on the one or more support frames 158. The one or more power supply units are protected by a protective cover 138. The one or more power supply units are configured to supply electric power to the functional components. In an illustrative embodiment, a power supply unit of the one or more power supply units associated with the rotating arm 108 and the angle arm assembly 110 is a Pro-Range Nickel Manganese Cobalt (NMC) 18650 11.1V 6000 mAh 3C 3S2P Li-Ion Battery Pack that is a Lithium NMC 18650-based rechargeable battery pack configured for high-energy applications. The Lithium NMC 18650-based rechargeable battery pack is configured with, but not limited to, at least one of: a nominal voltage of 11.1 Volts and a capacity of 6000 Milliampere-hour (mAh), with a 3C discharge rate, meaning the Lithium NMC 18650-based rechargeable battery pack may deliver up to 18A (3×6000 mAh). The Lithium NMC 18650-based rechargeable battery pack features a Battery Management System (BMS) for protection, a 4-pin Japan Solderless Terminal (JST) XH charge connector, and an Amass Nylon T discharge port. A maximum charging voltage is 12.6V, and a minimum discharging voltage is 8.7V, with a 1C charging current limit of 6A. The 3S2P configuration means the Lithium NMC 18650-based rechargeable battery pack comprises of three series and two parallel 18650 cells, balancing voltage and capacity.

In an illustrative embodiment, the power supply unit of the one or more power supply units employed for autonomous motion features is configured with, but not limited to, at least one of: a nominal capacity of 7800 mAh and a nominal voltage of 29.6V, resulting in a nominal energy of 231 Watt-hour (Wh), calculated as Capacity (Ah)×Voltage (V). The power supply unit requires a charging voltage of 33.6±0.05V and a charging current of 1950 milliamperes (mA), with a charging time of 5.0 hours using a Constant Current-Constant Voltage (CC-CV) method. The continuous discharge current is 1560 mA, while a peak discharge current reaches 25A. A discharge end voltage is 22V, and the power supply unit operates within a charge temperature range of 45 Degrees Celsius (°C) and a discharge temperature range of 60 °C.

In an illustrative embodiment, the first actuation unit 114 and the second actuation unit 116 are the direct current (DC) motors configured with, but not limited to, at least one of: no-load speed of 240 Revolutions Per Minute (RPM) and a rated speed of 225-230 RPM. The first actuation unit 114 and the second actuation unit 116 provide a rated torque of 63.9 Newton-centimetre (N-cm) and operate at a no-load current of 370 mA, while the full-load current and rated current are ≤3000 mA. The power rating of the first actuation unit 114 and the second actuation unit 116 is 13.34 W. The first actuation unit 114 and the second actuation unit 116 feature a shaft length of 27 mm and a shaft diameter of 8 mm. The first actuation unit 114 and the second actuation unit 116 rated RPM is 6000, with a gear ratio of 25:1. The dimensions of the first actuation unit 114 and the second actuation unit 116 are 122 mm (length)×70 mm (width)×70 mm (height), and weight is 480 g (grams).

In an exemplary embodiment, a display panel is operatively positioned in proximity to the one or more power supply units. The display panel is configured to display different messages to the one or more users, including, but not restricted to, at least one of: a level of the cleaning agent in the liquid tank 130, a charging level of the one or more power supply units, errors if there are any problems regarding hardware and the system, and the like. In an illustrative embodiment, the display panel is a blue Organic Light-Emitting Diode (OLED) display that communicates with the microcontroller unit 112 via an Inter-Integrated Circuit (I2C)/Inter-Integrated Communication (IIC) serial interface for easy integration in embedded systems.

In an exemplary embodiment, as shown in FIGS. 1H-1I, the angle arm assembly 110 rotates between 0 degree and 23 degrees to ensure thorough cleaning of a toilet surface associated with the diverse surfaces. The angle arm assembly 110 is configured to adapt to the curvature of the toilet surface (commode). The autonomous cleaning device 100 determines the surface characteristics, optimizing a cleaning trajectory. The autonomous cleaning device 100 ensures effective contact between the one or more cleaning equipment 132 and the toilet surface for consistent cleaning performance. The autonomous cleaning device 100 provides an efficient and hygienic solution for toilet maintenance.

The system may be configured with one or more computer vision models configured to detect and analyse at least one of: the shape, the orientation, and a seating area of the toilet surface in the first visual data for the optimal placement and alignment of the autonomous cleaning device 100 for the cleaning operations.

In an exemplary embodiment, FIGS. 1J-1K illustrate the positioning and an angular alignment of the autonomous cleaning device 100. FIGS. 1J-1K illustrate the angular range of movement, thereby allowing the autonomous cleaning device 100 to adjust precisely with specific target locations. The distances and the angles provided for positions A1 to A5, B1 to B5, C1 to C5, and D1 to D5 suggest a calculated alignment for effective coverage, ensuring optimal cleaning operations across the diverse surfaces.

A distance from X1 to X2 is 196.2 mm, and X3 to X4 is 192.5 mm. Table 1 and Table 2 indicate that the system accounts for positional deviations, with a tolerance of +5° for angular corrections. This flexibility ensures the autonomous cleaning device 100 may adapt to variations in real-world conditions, maintaining accuracy even when minor misalignments occur.

TABLE 1

| | | Distance | Angle |
|---|---|---|---|
| Category | Point | (mm) | (°) |
| Position—from arm (vertical | B1 | 156.4 | 19.56 |
| line) | | | |
| Position—from arm (vertical | B2 | 127.5 | 14.59 |
| line) | | | |
| Position—from arm (vertical | B3 | 98.8 | 10.35 |
| line) | | | |
| Position—from arm (vertical | B4 | 70.4 | 6.81 |
| line) | | | |
| Position—from arm (vertical | B5 | 32.7 | 2.93 |
| line) | | | |
| Position—from arm (vertical | D1 | 178 | 22.02 |
| line) | | | |
| Position—from arm (vertical | D2 | 161.3 | 18.21 |
| line) | | | |
| Position—from arm (vertical | D3 | 144.3 | 14.95 |
| line) | | | |
| Position—from arm (vertical | D4 | 141.6 | 13.49 |
| line) | | | |
| Position—from arm (vertical | D5 | 113.6 | 10.06 |
| line) | | | |
| From X1 to X2 | — | 196.2 | — |

TABLE 2

| | | Distance | Angle |
|---|---|---|---|
| Category | Point | (mm) | (°) |
| Position—from arm (vertical | C1 | 153.9 | 20.24 |
| line) | | | |
| Position—from arm (vertical | C2 | 125.1 | 14.98 |
| line) | | | |
| Position—from arm (vertical | C3 | 96.2 | 10.53 |
| line) | | | |
| Position—from arm (vertical | C4 | 65.6 | 6.53 |
| line) | | | |
| Position—from arm (vertical | C5 | 32.9 | 3.05 |
| line) | | | |
| Position—from arm (vertical | A1 | 154 | 20.25 |
| line) | | | |
| Position—from arm (vertical | A2 | 125.2 | 14.99 |
| line) | | | |
| Position—from arm (vertical | A3 | 96.3 | 10.54 |
| line) | | | |
| Position—from arm (vertical | A4 | 73.9 | 7.42 |
| line) | | | |
| Position—from arm (vertical | A5 | 36.2 | 3.36 |
| line) | | | |
| From X3 to X4 | — | 192.5 | — |

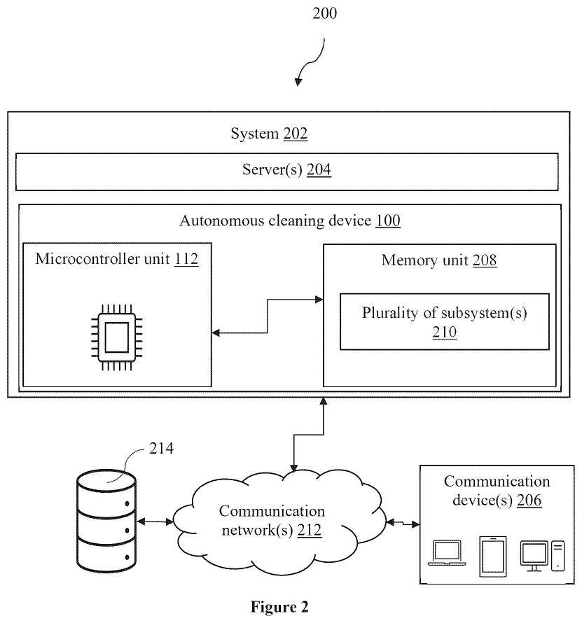

FIG. 2 illustrates an exemplary block diagram representation of a network architecture 200 depicting the system 202 for the autonomous AI-based cleaning of the diverse surfaces, in accordance with an embodiment of the present invention.

According to an exemplary embodiment of the disclosure, the network architecture 200 may include the system 202, one or more databases 214, and the one or more communication devices 206. The system 202, the one or more databases 214, and the one or more communication devices 206 may be communicatively coupled via one or more communication networks 212, ensuring seamless data transmission, processing, and decision-making. The system 202 acts as a central processing unit within the network architecture 200, responsible for the autonomous AI-based cleaning of the diverse surfaces. The system 202 is configured to execute a set of computer-readable instructions that control a plurality of subsystems 210.

In an exemplary embodiment, the system 202 comprises the one or more servers 204. The one or more servers 204 may comprise a combination of discrete components, an integrated circuit, an application-specific integrated circuit, a field-programmable gate array, a digital signal processor, or other suitable hardware. The “software” may comprise one or more objects, agents, threads, lines of code, subroutines, separate software applications, two or more lines of code, or other suitable software structures operating in one or more software applications or the microcontroller unit 112.

The one or more servers 204 are configured to process and manage data within the system 202. The one or more servers 204 may be digital computing devices, cloud-based computing platforms, or networked infrastructures. The one or more servers 204 may include, but are not limited to, at least one of: data centres, cloud computing instances, high-performance computing clusters, and dedicated artificial intelligence (AI) processing servers that enable seamless communication and data exchange with the system 202.

In an exemplary embodiment, the one or more communication devices 206 are configured to enable the one or more users to interact with the system 202 and the autonomous cleaning device 100. The one or more communication devices 206 may be digital devices, computing devices, and/or networks. The one or more communication devices 206 may include, but not limited to, a mobile device, a smartphone, a personal digital assistant (PDA), a tablet computer, a phablet computer, a wearable computing device, a laptop, a desktop, and the like.

The autonomous cleaning device 100 may comprise the microcontroller unit 112 and the memory unit 208. The memory unit 208 is operatively connected to the microcontroller unit 112. The memory unit 208 comprises the set of computer-readable instructions in the form of the plurality of subsystems 210, configured to be executed by the microcontroller unit 112.

In an exemplary embodiment, the microcontroller unit 112 may include, for example, a microprocessor, a microcomputer, a microcontroller, a digital signal processor, a central processing unit, a state machine, a logic circuit, and/or any device that manipulates data or signals based on operational instructions. Among other capabilities, the microcontroller unit 112 may fetch and execute computer-readable instructions in the memory unit 208 operationally coupled with the system 202 for performing tasks such as data processing, input/output processing, and/or any other functions. Any reference to a task in the present disclosure may refer to an operation being or that may be performed on data. The microcontroller unit 112 is a high-performance processor capable of handling large volumes of data and complex computations.

In an exemplary embodiment, the one or more databases 214 may be configured to store and manage data related to various aspects of the system 202. The one or more databases 214 may store at least one of, but not limited to, information of the diverse surfaces, sensor data, the first visual data, second visual data, information associated with the autonomous cleaning device 100, any other information necessary for the functionality and optimization of the system 202, and the like. The one or more databases 214 serve as a centralized repository for critical data elements that are integral to the secure operation of the system 202, enabling the autonomous AI-based cleaning of the diverse surfaces. The one or more databases 214 enable the system 202 to dynamically retrieve, analyse, and update the stored data in real-time, for the autonomous AI-based cleaning of the diverse surfaces. The one or more databases 214 may include different types of databases such as, but not limited to, relational databases (e.g., Structured Query Language (SQL) databases such as PostgresDB and Oracle® databases), non-Structured Query Language (NoSQL) databases (e.g., MongoDB, Cassandra), time-series databases (e.g., InfluxDB), an OpenSearch database, object storage systems (e.g., Amazon® S3), cloud databases, and the like.

In an exemplary embodiment, the one or more communication networks 212 may be, but not limited to, a wired communication network and/or a wireless communication network, a local area network (LAN), a wide area network (WAN), a Wireless Local Area Network (WLAN), a metropolitan area network (MAN), a telephone network, such as the Public Switched Telephone Network (PSTN) or a cellular network, an intranet, the Internet, a fibre optic network, a satellite network, a cloud computing network, a combination of networks, and the like. The wired communication network may comprise, but not limited to, at least one of: Ethernet connections, Fiber Optics, Power Line Communications (PLCs), Serial Communications, Coaxial Cables, Quantum Communication, Advanced Fiber Optics, Hybrid Networks, and the like. The wireless communication network may comprise, but not limited to, at least one of: wireless fidelity (wi-fi), cellular networks (including fourth generation (4G) technologies and fifth generation (5G) technologies), Bluetooth®, ZigBee®, long-range wide area network (LoRaWAN), satellite communication, radio frequency identification (RFID), 6G (sixth generation) networks, advanced IoT protocols, mesh networks, non-terrestrial networks (NTNs), near field communication (NFC), and the like.

In an exemplary embodiment, the system 202 may be implemented by way of a single device or a combination of multiple devices that may be operatively connected or networked together. The system 202 may be implemented in hardware or a suitable combination of hardware and software.

Though few components and the plurality of subsystems 210 are disclosed in FIG. 1, there may be additional components and subsystems which is not shown, such as, but not limited to, ports, routers, repeaters, firewall devices, network devices, the one or more databases 214, network attached storage devices, assets, machinery, instruments, facility equipment, emergency management devices, image capturing devices, any other devices, and combination thereof. The person skilled in the art should not be limiting the components/subsystems shown in FIG. 1. Although FIG. 1 illustrates the system 202, and the one or more communication devices 206 connected to the one or more databases 214, one skilled in the art can envision that the system 202, and the one or more communication devices 206 may be connected to several user devices located at various locations and several databases via the one or more communication networks 212.

Those of ordinary skilled in the art will appreciate that the hardware depicted in FIG. 1 may vary for particular implementations. For example, other peripheral devices such as an optical disk drive and the like, the local area network (LAN), the wide area network (WAN), wireless (e.g., wireless-fidelity (Wi-Fi)) adapter, graphics adapter, disk controller, input/output (I/O) adapter also may be used in addition or place of the hardware depicted. The depicted example is provided for explanation only and is not meant to imply architectural limitations concerning the present disclosure.

Those skilled in the art will recognize that, for simplicity and clarity, the full structure and operation of all data processing systems suitable for use with the present disclosure are not being depicted or described herein. Instead, only so much of the system 202 as is unique to the present disclosure or necessary for an understanding of the present disclosure is depicted and described. The remainder of the construction and operation of the system 202 may conform to any of the various current implementations and practices that were known in the art.

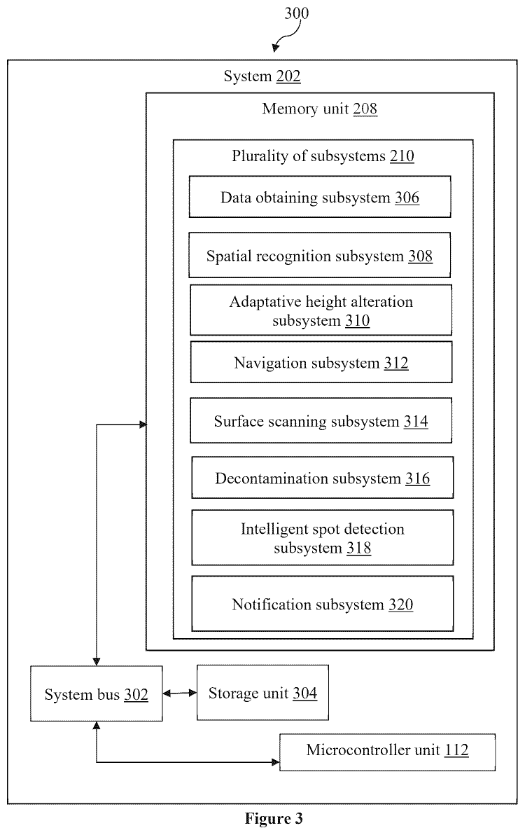

FIG. 3 illustrates an exemplary block diagram representation 300 of the system 202 as shown in FIG. 1 for the autonomous AI-based cleaning of the diverse surfaces, in accordance with an embodiment of the present invention.

In an exemplary embodiment, the microcontroller unit 112, the memory unit 208, and the storage unit 204 are communicatively coupled through a system bus 302 or any similar mechanism. The system bus 302 functions as the central conduit for data transfer and communication between the microcontroller unit 112, the memory unit 208, and the storage unit 204. The system bus 302 facilitates the efficient exchange of information and instructions, enabling the coordinated operation of the system 202. The system bus 302 may be implemented using various technologies, including but not limited to, parallel buses, serial buses, and high-speed data transfer interfaces such as, but not limited to, at least one of a: universal serial bus (USB), peripheral component interconnect express (PCIe), and similar standards.

In an exemplary embodiment, the memory unit 208 is operatively connected to the microcontroller unit. 112. The memory unit 208 comprises the plurality of subsystems 210 in the form of the set of computer-readable instructions executable by the microcontroller unit 112. The plurality of subsystems 210 comprises a data obtaining subsystem 306, a spatial recognition subsystem 308, an adaptative height alteration subsystem 310, a navigation subsystem 312, a surface scanning subsystem 314, a decontamination subsystem 316, an intelligent spot detection subsystem 318, and a notification subsystem 320.

The microcontroller unit 112, as used herein, means any type of computational circuit, such as, but not limited to, a microprocessor unit, a complex instruction set computing microprocessor unit, a reduced instruction set computing microprocessor unit, a very long instruction word microprocessor unit, a explicitly parallel instruction computing microprocessor unit, a graphics processing unit, a digital signal processing unit, or any other type of processing circuit. The microcontroller unit 112 may also include embedded controllers, such as generic or programmable logic devices or arrays, application-specific integrated circuits, single-chip computers, and the like.

The memory unit 208 may be the non-transitory volatile memory and the non-volatile memory. The memory unit 208 may be coupled to communicate with the microcontroller unit 112, such as being a computer-readable storage medium. The microcontroller unit 112 may execute machine-readable instructions and/or source code stored in the memory unit 208. A variety of machine-readable instructions may be stored in and accessed from the memory unit 208. The memory unit 208 may include any suitable elements for storing data and machine-readable instructions, such as read-only memory, random access memory, erasable programmable read-only memory, electrically erasable programmable read-only memory, a hard drive, a removable media drive for handling compact disks, digital video disks, diskettes, magnetic tape cartridges, memory cards, and the like. In the present embodiment, the memory unit 208 includes the plurality of subsystems 210 stored in the form of the set of computer-readable instructions on any of the above-mentioned storage media and may be in communication with and executed by the microcontroller unit 112.

The storage unit 204 may be a cloud storage or the one or more databases 214 such as those shown in FIG. 1. The storage unit 204 may store, but not limited to, recommended course of action sequences dynamically generated by the system 202. The action sequences comprise data obtaining, spatial recognition, adaptative height alteration, providing navigation, surface scanning, performing decontamination, intelligent spot detection, providing notification, and the like. Additionally, the storage unit 204 may retain previous action sequences for comparison and future reference, enabling continuous refinement of the system 202 over time. The storage unit 204 may be any kind of database such as, but not limited to, relational databases, dedicated databases, dynamic databases, monetized databases, scalable databases, cloud databases, distributed databases, any other databases, and a combination thereof.

In an exemplary embodiment, the data obtaining subsystem 306 is configured to obtain the sensor data to initiate the cleaning process on the diverse surfaces. This ensures that the autonomous cleaning device 100 adapts the cleaning operations based on real-time conditions. This enhances efficiency by enabling targeted cleaning based on actual surface requirements rather than pre-programmed routines. In another exemplary embodiment, the system 202 is configured to employ a TensorFlow data model. The TensorFlow data model is trained on the images associated with the first visual data and the second visual data to distinguish between normal images and anomalous images. The first visual data and the second visual data comprise at least one of: images and videos including, but not limited to, at least one of: the diverse surfaces, the one or more obstacles, contamination patterns, liquid spills, dust accumulation, stain formations, bacterial growth indicators, surface texture variations, and the like.

In an exemplary embodiment, the spatial recognition subsystem 308 is configured to adjust a cleaning approach according to the one or more spatial characteristics of the environment. The spatial recognition subsystem 308 is configured to trigger the first actuation unit 114 to rotate the visual data capturing unit, which records the first visual data of the diverse surfaces. The first visual data is analysed to determine the one or more spatial characteristics. The one or more spatial characteristics may comprise, but not restricted to, at least one of: the coordinates of the targeted surface, the height of the targeted surface, confined spaces, edges, and corners on the diverse surfaces, surface material type, surface texture, and the like. By accurately recognizing the one or more spatial characteristics, the system 202 ensures that the one or more cleaning equipment 132 apply appropriate pressure, movement patterns, and the cleaning agent suited to the specific surface conditions.

In an exemplary embodiment, the adaptative height alteration subsystem 310 is configured to dynamically adjust the height and movement of the autonomous cleaning device 100 based on the real-time one or more spatial characteristics. The adaptative height alteration subsystem 310 interacts with a third actuation unit 118 (motor) and a fourth actuation unit 120 (motor) associated with the plurality of actuation units, which control the plurality of articulated shafts 102 and the wheel assembly, respectively. By altering the height of the autonomous cleaning device 100 using the third actuation unit 118 and the fourth actuation unit 120, the adaptative height alteration subsystem 310 ensures optimal positioning for effective cleaning, particularly on uneven, elevated, and confined surfaces of the diverse surfaces. The adaptative height alteration subsystem 310 may also regulate the speed of the autonomous cleaning device 100 by controlling the fourth actuation unit 120, allowing for smooth navigation across varied terrains, obstacles, and surface transitions.

The plurality of articulated shafts 102 provides the necessary flexibility and precision to alter the height while maintaining stability. Additionally, by adjusting the wheel assembly, the adaptative height alteration subsystem 310 optimizes movement patterns to ensure efficient cleaning on at least one of: flat floors, inclined ramps, textured surfaces, and hard-to-reach corners.

In an exemplary embodiment, the navigation subsystem 312 is configured to generate obstacle avoidance data and determine a road map (optimal movement path) to the targeted surface. The navigation subsystem 312 processes at least one of: the first visual data, the depth perception data, and the IR data through, but not limited to, at least one of: one or more predictive motion models and one or more deep reinforcement learning models. The one or more predictive motion models refer to computational frameworks that anticipate the movement of the autonomous cleaning device 100 and nearby one or more obstacles by analysing past motion patterns and environmental inputs. The one or more predictive motion models leverage at least one of: historical movement data, obstacle interaction scenarios, and Simultaneous Localization and Mapping (SLAM) datasets (includes spatial and motion data used for real-time mapping and localization) to refine the accuracy. Meanwhile, the one or more deep reinforcement learning models improve navigation by continuously learning from at least one of: Simulated robotic movement datasets (includes synthetic motion and interaction data to train and optimise robotic navigation models in virtual environments) and Reinforcement learning-based training simulations, enabling adaptive responses to real-world one or more obstacles. The Reinforcement learning-based training simulations involve the virtual environments where models iteratively learn optimal navigation strategies by interacting with dynamic one or more obstacles.

Once the obstacle avoidance data is generated, the navigation subsystem 312 processes the obstacle avoidance data along with the first visual data to develop the road map. The road map is generated by employing, but not limited to, at least one of: one or more path planning models, one or more reinforcement learning models, one or more motion planning models, and the like. The one or more path planning models determine the optimal road map from a current position of the autonomous cleaning device 100 to the targeted surface while avoiding the one or more obstacles. At least one of: the one or more path planning models, the one or more reinforcement learning models, and the one or more motion planning models are trained on dynamic navigation datasets. The dynamic navigation datasets may comprise, but not constrained to, at least one of: a) terrain recognition data that adjusts motion parameters based on the one or more spatial characteristics and b) obstacle-rich real-world scenario data for adaptive movement decision-making.

The one or more motion planning models calculate real-time movements by analysing environmental constraints and optimizing speed and orientation. To enhance adaptability, the navigation subsystem 312 incorporates the one or more reinforcement learning models trained on the obstacle-rich real-world scenario data. The one or more reinforcement learning models optimize decision-making by learning from trial and error, improving how the autonomous cleaning device 100 reacts to new and unexpected one or more obstacles. At least one of: the one or more path planning models, the one or more reinforcement learning models, and the one or more motion planning models enable the autonomous cleaning device 100 to dynamically adjust the trajectory. This ensures seamless navigation, collision-free movement, and precise cleaning coverage across diverse environments.

In an exemplary embodiment, the surface scanning subsystem 314 is configured to assess surface conditions using one or more Convolutional Neural Networks (CNNs). The one or more CNNs are employed for image recognition and analysis. The one or more CNNs process the first visual data and the sensor data to extract at least one of: one or more geometrical features and contamination data of the targeted surface. The one or more geometrical features of the targeted surface may comprise, but not limited to, at least one of: surface curvature profile, edge contours, depth variations, surface area dimensions, inclination angles, surface irregularities, symmetry and shape classification, and the like. The one or more geometrical features assist the autonomous cleaning device 100 in identifying the diverse surface as one of: flat, concave, convex, irregular, and configured with sharp boundaries, ensuring the appropriate cleaning mechanism is selected for optimal coverage.

The contamination data may comprise, but not be restricted to, at least one of: the contaminants, surface stain intensity, bacterial presence data, chemical residue detection, organic waste detection, liquid spill analysis, dust and the particulate accumulation, Odor intensity data, and the like. To accurately classify and interpret the contamination data, the one or more CNNs are trained using image-based contamination datasets. The image-based contamination datasets may include, but not constricted to, at least one of: a) labelled contamination datasets for distinguishing different contaminants. The labelled contamination datasets are employed for classifying the contamination data and the one or more geometrical features, b) supervised learning datasets to predict a required cleaning intensity based on contamination severity. The supervised learning datasets are employed for analysing the contamination data to predict the cleaning intensity, and c) labelled geometric feature datasets for analysing the one or more geometrical features. The labelled geometric feature datasets allow the system 202 to differentiate between at least one of: concave surfaces, convex surfaces, irregular surfaces, boundaries, edges, corners, and surface depth variations. By leveraging the image-based contamination datasets, the one or more CNNs improve the ability to recognize patterns and adapt the cleaning process accordingly.

In an exemplary embodiment, the decontamination subsystem 316 is configured to select at least one of: the corresponding cleaning equipment 132 from the one or more cleaning equipment 132 and a quantity of a cleaning agent based on the contamination data. The decontamination subsystem 316 leverages the one or more CNNs to analyse contamination types and determine the corresponding cleaning equipment 132. Additionally, the decontamination subsystem 316 calculates the optimal quantity of the cleaning agent, ensuring effective cleaning while minimizing waste.

To maintain a consistent contact between the corresponding cleaning equipment 132 and the targeted surface, the decontamination subsystem 316 employs a gyroscopic stabilization mechanism. The gyroscopic stabilization mechanism adjusts the position of the corresponding cleaning equipment 132 by activating the fifth actuation unit 122 for the cleaning equipment assembly and a sixth actuation unit 124 associated with the plurality of actuation units for the liquid tank 130. By analysing the one or more geometrical features, the decontamination subsystem 316 ensures uniform cleaning pressure and coverage on the diverse surfaces. In an illustrative embodiment, the fifth actuation unit 122 is a stepper motor configured to increase the height of the cleaning equipment assembly along with the corresponding cleaning equipment 132. A link motor 148 is a mechanical structure that assists in moving the cleaning equipment assembly. A first lead screw 144 is configured to expand and contract the cleaning equipment assembly. The first lead screw 144 is configured with the dimensions Ø8 millimetres (mm) x200 (2 mm Pitch). The first lead screw 144 is controlled by the fifth actuation unit 122. A coupling 146 is configured to hold all the parts of the cleaning equipment assembly.

In an illustrative embodiment, the sixth actuation unit 124 is the stepper motor configured to move the liquid tank 130 near to the target surface for optimal cleaning agent dispensing and to avoid spillage of the cleaning agent. A second lead screw 150 is configured with the dimensions of Ø8×180 (2 mm Pitch) to expand and contract the liquid tank 130 for releasing the cleaning agent on the one or more cleaning equipment 132. The second lead screw 150 is controlled by the seventh actuation unit 126.

Furthermore, the decontamination subsystem 316 triggers a seventh actuation unit 126 of the plurality of actuation units associated with the liquid tank assembly 174 to dispense the determined quantity of the cleaning agent on to the corresponding cleaning equipment 132, optimizing cleaning effectiveness based on contamination severity. In an illustrative embodiment, the seventh actuation unit 126 is the DC motor. The seventh actuation unit 126 is configured to release a gear 152 so that the cleaning agent flows into a lower part of the liquid tank 130. The gear 152 is a rotating mechanical component driven by the seventh actuation unit 126. The gear 152 eases the opening of the liquid tank 130. A connector 154 is configured to hold the seventh actuation unit 126 onto the liquid tank 130. A slider-gear 156 is a part of a linear motion mechanism, where the slider-gear 156 is operatively connected to the gear 152. The slider-gear 156 converts the rotational motion into linear displacement. The slide-gear 156 is configured to provide a smooth and controlled flow of the cleaning agent towards the one or more cleaning equipment 132 via a nozzle 136.

The decontamination subsystem 316 is configured to initiate a first-round cleaning operation using the pre-defined sequential patterns. The decontamination subsystem 316 is configured to trigger the cleaning equipment actuation unit 128 for initiating the first-round of cleaning based on the contamination data. By dynamically adjusting the cleaning process, the decontamination subsystem 316 enhances cleaning efficiency, making the autonomous cleaning device 100 suitable for, but not limited to, at least one of: autonomous facility maintenance, industrial cleaning, medical sanitation applications, and the like.

In an exemplary embodiment, the intelligent spot detection subsystem 318 is configured to enhance the effectiveness of the autonomous cleaning device 100 by identifying at least one of: residual contaminants and particulate accumulation after the first-round of cleaning. The intelligent spot detection subsystem 318 employs at least one of: the visual data capturing unit and the one or more chemical sensors. The visual data capturing unit is configured to capture the second visual data, which is compared against the first visual data (captured before cleaning) to assess changes in surface cleanliness. The intelligent spot detection subsystem 318 may detect remaining dust, stains, bacterial presence, and chemical residues using the one or more chemical sensors, ensuring that the targeted surface is cleaned to optimal hygienic standards. If the contaminants persist, the intelligent spot detection subsystem 318 triggers a second-round of cleaning for targeted spot cleaning.

To quantitatively evaluate cleanliness, the intelligent spot detection subsystem 318 calculates a cleanliness score by comparing the first visual data with the second visual data. This assessment is performed using at least one of: difference mapping models and threshold-based scoring procedures. The difference mapping models analyse pixel-level variations between the first visual data with the second visual data, highlighting areas where the contamination remains. Meanwhile, the threshold-based scoring procedures assign the cleanliness score based on predefined parameters such as, but not limited to, at least one of: stain intensity, particulate density, and bacterial detection levels. This automated evaluation allows the system 202 to dynamically adjust cleaning intensity for optimal results.

By integrating real-time cleanliness assessment with adaptive cleaning responses, the intelligent spot detection subsystem 318 ensures efficient and resource-conscious cleaning operations. Instead of performing unnecessary repeated cleaning cycles, the system 202 targets only the targeted surfaces that require further cleaning, thereby minimizing energy and cleaning agent consumption.

In an exemplary embodiment, the notification subsystem 320 is configured to provide real-time updates and the one or more alert notifications to the one or more users regarding one or more operational parameters to ensure seamless functionality and maintenance. The one or more operational parameters may include, but not limited to, at least one of: a) the cleaning statistics, which provide insights into performance metrics such as area covered, time taken, and effectiveness based on the cleanliness score, b) cleaning agent refilling status to prevent interruptions due to insufficient cleaning agent levels, c) power level status, sending timely alerts to prompt charging before the autonomous cleaning device 100 runs out of power, d) system performance diagnostics and sensor calibration alerts, assisting the one or more users in detecting potential hardware and system issues that may affect cleaning efficiency, such as misaligned sensors and degraded actuator performance, e) real-time operational status feature enables the one or more users to remotely track the movement, cleaning patterns, and efficiency via IoT-based facility management platforms, and f) model update alerts ensure that the one or more users are informed about the availability of new AI-driven improvements, allowing for seamless over-the-air (OTA) updates.

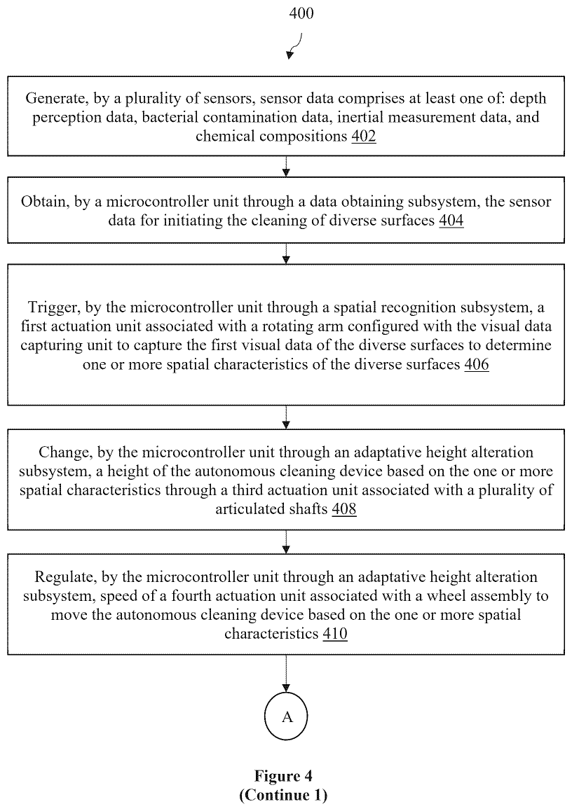

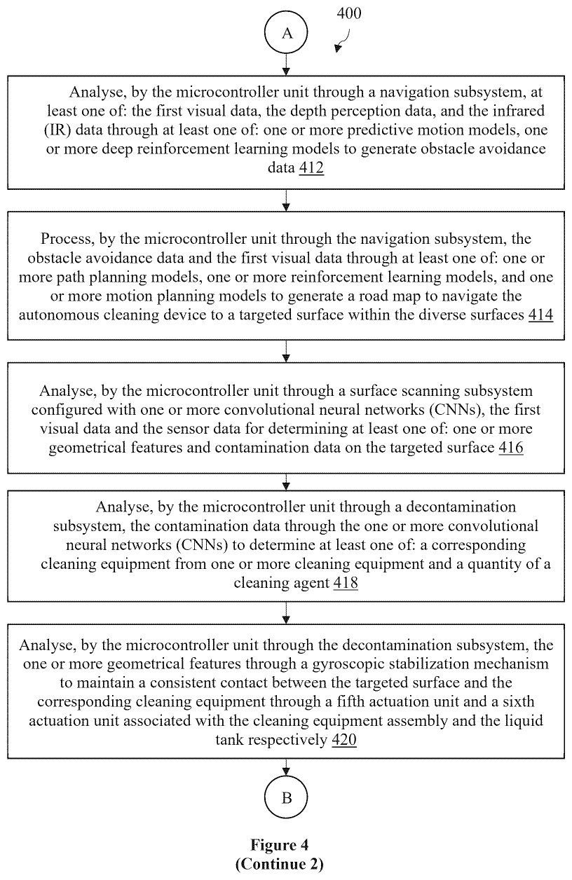

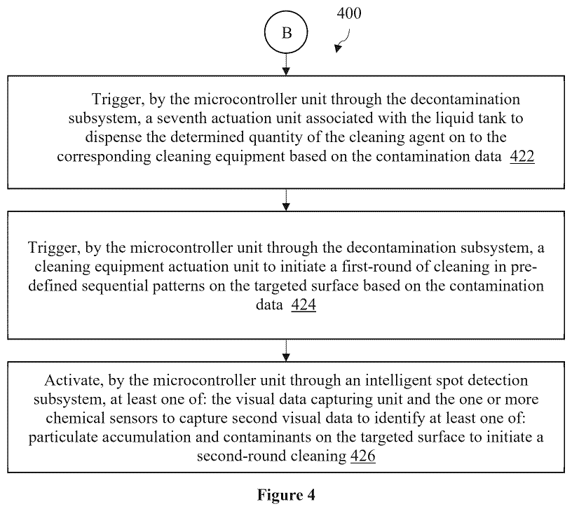

FIG. 4 illustrates an exemplary flow diagram representation depicting a method 400 for the autonomous AI-based cleaning of the diverse surfaces, in accordance with an embodiment of the present invention.

According to an exemplary embodiment of the disclosure, the method 400 for the autonomous AI-based cleaning of the diverse surfaces is disclosed. At step 402, the method 400 involves generating the sensor data using the plurality of sensors, capturing key environmental parameters. The sensor data may include, but not restricted to, at least one of: the depth perception data for spatial analysis, the bacterial contamination data for hygiene assessment, the inertial measurement data for motion tracking, the chemical compositions for detecting surface residues, and the like.

At step 404, the method 400 involves obtaining the sensor data by the microcontroller unit through the data obtaining subsystem to initiate the cleaning of the diverse surfaces. This step 404 ensures that the autonomous cleaning device accurately evaluates surface conditions before activating the cleaning mechanisms.

At step 406, the method 400 involves triggering the first actuation unit through the spatial recognition subsystem, controlled by the microcontroller unit. The first actuation unit operates the rotating arm, which is equipped with the visual data capturing unit to capture the first visual data of the diverse surfaces. The captured first visual data is then analysed to determine the one or more spatial characteristics of the diverse surfaces.

At step 408, the method 400 involves adjusting the height of the autonomous cleaning device based on the identified one or more spatial characteristics. This adjustment is performed by the microcontroller unit through the adaptative height alteration subsystem. The adaptative height alteration subsystem triggers the third actuation unit, which is linked to the plurality of articulated shafts, allowing the autonomous cleaning device to modify the height dynamically.

At step 410, the method 400 involves regulating the movement speed of the autonomous cleaning device based on the identified one or more spatial characteristics. This is accomplished by the microcontroller unit through the adaptative height alteration subsystem, which triggers the fourth actuation unit associated with the wheel assembly. By dynamically adjusting the speed, the autonomous cleaning device ensures optimal navigation, stability, and precision while cleaning the diverse surfaces of varying elevations, confined spaces, and complex structures.

At step 412, the method 400 involves analysing at least one of: the first visual data, the depth perception data, and the IR data using the navigation subsystem. The microcontroller unit processes at least one of: the first visual data, the depth perception data, and the IR data through at least one of: the one or more predictive motion models, the one or more deep reinforcement learning models to generate the obstacle avoidance data. This enables the autonomous cleaning device to detect and navigate around the one or more obstacles efficiently, ensuring seamless movement and optimized cleaning performance in dynamic environments

At step 414, the method 400 processing the obstacle avoidance data and the first visual data using the navigation subsystem. The microcontroller unit utilizes at least one of: the one or more path planning models, the one or more reinforcement learning models, and the one or more motion planning models to generate the road map. The road map guides the autonomous cleaning device in navigating efficiently toward the targeted surface within the diverse surfaces, ensuring precise and adaptive movement for optimal cleaning.

At step 416, the method 400 involves analysing the first visual data and the sensor data using the surface scanning subsystem, which is configured with the one or more CNNs. The microcontroller unit processes the first visual data and the sensor data to determine at least one of: the one or more geometrical features and the contamination data on the targeted surface.

At step 418, the method 400 involves analysing the contamination data using the decontamination subsystem, which is processed by the microcontroller unit through the one or more CNNs. This analysis determines at least one of: the corresponding cleaning equipment from the one or more cleaning equipment and the quantity of the cleaning agent. By leveraging machine learning-based contamination assessment, the system ensures optimal cleaning efficiency while minimizing waste.

At step 420, the method 400 involves analysing the one or more geometrical features using the decontamination subsystem, processed by the microcontroller unit through the gyroscopic stabilization mechanism. This analysis ensures the consistent contact between the targeted surface and the corresponding cleaning equipment by triggering the fifth actuation unit associated with the cleaning equipment assembly and the sixth actuation unit linked to the liquid tank. This precise stabilization mechanism enhances cleaning efficiency by adapting to surface irregularities.

At step 422, the method 400 involves triggering the seventh actuation unit, via the microcontroller unit through the decontamination subsystem, to dispense the determined quantity of the cleaning agent on to the corresponding cleaning equipment based on the contamination data. This ensures precise application of the cleaning agent, optimising cleaning effectiveness while minimizing the waste.

At step 424, the method 400 involves triggering the cleaning equipment actuation unit, via the microcontroller unit through the decontamination subsystem, to initiate the first-round of cleaning in the predefined sequential patterns on the targeted surface based on the contamination data. This ensures systematic and efficient cleaning tailored to the specific contamination detected.

At step 426, the method 400 involves activating the intelligent spot detection subsystem, via the microcontroller unit, to trigger at least one of: the visual data capturing unit and the one or more chemical sensors to capture the second visual data. The second visual data is configured to identify at least one of: the particulate accumulation and the contaminants on the targeted surface, enabling the initiation of the second-round of cleaning if necessary.

Numerous advantages of the present disclosure may be apparent from the discussion above. In accordance with the present disclosure, the system for the autonomous AI-based cleaning of the diverse surfaces is disclosed. The autonomous cleaning device is trained for self-plugging into a power socket to get charged. The autonomous cleaning device is also configured to perform the cleaning operations when manually placed in a certain position. The system may identify and act on the anomalies that are present on the diverse surfaces. The system considers pressure-sensitive movement to optimize surface contact.

While specific language has been used to describe the invention, any limitations arising on account of the same are not intended. As would be apparent to a person skilled in the art, various working modifications may be made to the method in order to implement the inventive concept as taught herein.

The figures and the foregoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, order of processes described herein may be changed and are not limited to the manner described herein. Moreover, the actions of any flow diagram need not be implemented in the order shown; nor do all of the acts need to be necessarily performed. Also, those acts that are not dependent on other acts may be performed in parallel with the other acts. The scope of embodiments is by no means limited by these specific examples.

I/We claim:

1. A system for autonomous artificial intelligence-based (AI-based) cleaning of diverse surfaces, comprising:
an autonomous cleaning device comprising:
a plurality of articulated shafts comprises a proximal end and a distal end, configured to change a height of the autonomous cleaning device based on the diverse surfaces,
the proximal end operatively connected to one or more support frames; and
the distal end of each articulated shaft of the plurality of articulated shafts connected to a wheel assembly;
a fixture comprises a first surface and a second surface, operatively connected to the one or more support frames, wherein the fixture comprising:
a plurality of sensors operatively positioned on at least one of: the first surface and the second surface, configured to generate sensor data comprises at least one of: depth perception data, bacterial contamination data, inertial measurement data, and chemical compositions;
a rotating arm protruding from the second surface and connected to a first actuation unit operatively positioned on the first surface,
the rotating arm configured with a holder to hold a visual data capturing unit at a defined angle; and
the rotating arm configured to rotate the visual data capturing unit in a defined direction at a time of capturing first visual data of the diverse surfaces;
an angle arm assembly pivotally connected to a free end of the rotating arm through a second actuation unit, configured to change a position of at least one of:
a liquid tank and a cleaning equipment assembly at the defined angle to reach the diverse surfaces;
a microcontroller unit operatively positioned on the first surface;
a memory unit operatively connected to the microcontroller unit, wherein the memory unit comprises a set of computer-readable instructions in form of a plurality of subsystems, configured to be executed by the microcontroller unit, wherein the plurality of subsystems comprises:
a data obtaining subsystem configured to obtain the sensor data for initiating the cleaning of the diverse surfaces;
a spatial recognition subsystem configured to trigger the first actuation unit to actuate the rotating arm to capture the first visual data of the diverse surfaces for determining one or more spatial characteristics of the diverse surfaces;
an adaptative height alteration subsystem configured to trigger a third actuation unit, and a fourth actuation unit associated with the plurality of articulated shafts and the wheel assembly respectively to at least one of:
change the height of the autonomous cleaning device based on the one or more spatial characteristics; and
regulate speed of the fourth actuation unit to move the autonomous cleaning device based on the one or more spatial characteristics;
a navigation subsystem configured to:
generate obstacle avoidance data by analysing at least one of: the first visual data, the depth perception data, and the infrared (IR) data through at least one of: one or more predictive motion models, one or more deep reinforcement learning models; and
generate a road map to navigate the autonomous cleaning device to a targeted surface within the diverse surfaces based on processing the obstacle avoidance data and the first visual data through at least one of: one or more path planning models, one or more reinforcement learning models, and one or more motion planning models;
a surface scanning subsystem configured with one or more convolutional neural networks (CNNs) to analyse the first visual data and the sensor data for determining at least one of: one or more geometrical features and contamination data on the targeted surface;
a decontamination subsystem configured to:
determine at least one of: a corresponding cleaning equipment from one or more cleaning equipment and a quantity of a cleaning agent, based on analysing the contamination data through the one or more convolutional neural networks (CNNs);
maintain a consistent contact between the targeted surface and the corresponding cleaning equipment by triggering a fifth actuation unit and a sixth actuation unit associated with the cleaning equipment assembly and the liquid tank respectively by analysing the one or more geometrical features through a gyroscopic stabilization mechanism;
trigger a seventh actuation unit associated with the liquid tank to dispense the determined quantity of the cleaning agent on to the corresponding cleaning equipment based on the contamination data; and
trigger a cleaning equipment actuation unit for initiating a first-round of cleaning in pre-defined sequential patterns on the targeted surface based on the contamination data; and
an intelligent spot detection subsystem configured to activate at least one of: the visual data capturing unit and the one or more chemical sensors to capture second visual data to identify at least one of: particulate accumulation and contaminants on the targeted surface to initiate a second-round cleaning.

2. The system as claimed in claim 1, wherein the plurality of articulated shafts is selected from a group comprises at least one of: telescopic shafts, hydraulic shafts, pneumatic shafts, lead screw-driven shafts, rack and pinion shafts, scissor lift shafts, ball screw shafts, and electromechanical actuator shafts.

3. The system as claimed in claim 1, wherein the plurality of sensors comprises at least one of:
an obstacle detection unit configured to generate the depth perception data by computing a distance between the autonomous cleaning device and one or more obstacles for detecting the one or more obstacles on the diverse surfaces;
an inertial measurement unit (IMU) configured to determine acceleration and orientation of the autonomous cleaning device for generating the inertial measurement data;
at least one of: one or more infrared (IR) sensors and one or more ultraviolet (UV) sensors configured to generate the bacterial contamination data based on determining bacterial contamination levels on the targeted surface before and after cleaning;
one or more ultrasonic sensors configured to generate the one or more geometrical features of the targeted surface; and
the one or more chemical sensors configured to detect the chemical compositions on the targeted surface for dispensing an optimised quantity of the cleaning agent.

4. The system as claimed in claim 1, wherein the cleaning equipment assembly comprises at least one of: a cleaning equipment holder, the cleaning equipment actuation unit, and the one or more cleaning equipment,
the cleaning equipment holder operatively engage with the fifth actuation unit, configured to hold the cleaning equipment actuation unit;
the one or more cleaning equipment operatively connected to the cleaning equipment actuation unit for cleaning in the pre-defined sequential patterns,
the pre-defined sequential patterns comprise at least one of: a zig-zag pattern, a circular pattern, a radial pattern, a spiral pattern, a grid pattern, and a randomized adaptive pattern; and
the one or more cleaning equipment comprises at least one of: a soft bristle brush, a hard bristle brush, an antimicrobial brush, a microfiber cleaning pad, a rotating sponge head, a silicone scraper, an electrostatic dusting pad, and an ultraviolet (UV) sterilization module.

5. The system as claimed in claim 1, wherein
the one or more spatial characteristics comprise at least one of: coordinates of the targeted surface, height of the targeted surface, confined spaces, edges, and corners on the diverse surfaces, surface material type, and surface texture;
the one or more geometrical features of the targeted surface comprise at least one of: surface curvature profile, edge contours, depth variations, surface area dimensions, inclination angles, surface irregularities, and symmetry and shape classification; and
the contamination data comprises at least one of: the contaminants, surface stain intensity, bacterial presence data, chemical residue detection, organic waste detection, liquid spill analysis, dust and the particulate accumulation, and Odor intensity data.

6. The system (202) as claimed in claim 1, wherein
at least one of: the one or more predictive motion models, the one or more deep reinforcement learning models are trained on at least one of: historical movement data, obstacle interaction scenarios, Simulated robotic movement datasets, Reinforcement learning-based training simulations, Simultaneous Localization and Mapping (SLAM) datasets;
at least one of: the one or more path planning models, the one or more reinforcement learning models, and the one or more motion planning models are trained on dynamic navigation datasets,
the dynamic navigation datasets comprise at least one of:
terrain recognition data for altering motion parameters based on the one or more spatial characteristics; and
obstacle-rich real-world scenario data for adaptive movement decision-making; and
the one or more convolutional neural networks (CNNs) trained on image-based contamination datasets, wherein training includes at least one of:
labelled contamination datasets for classifying the contamination data and the one or more geometrical features;
supervised learning datasets for analysing the contamination data to predict a cleaning intensity; and
labelled geometric feature datasets for analysing the one or more geometrical features to recognize at least one of: concave surfaces, convex surfaces, irregular surfaces, boundaries, edges, corners, and surface depth variations.

7. The system as claimed in claim 1, wherein the intelligent spot detection subsystem configured to compare the first visual data with the second visual data to quantitatively assess a cleanliness score and adapt the cleaning intensity accordingly,
the cleanliness score is determined based on processing the first visual data and the second visual data through at least one of: difference mapping models and threshold-based scoring procedures.

8. The system as claimed in claim 1, wherein the plurality of subsystems comprises:
a notification subsystem configured to provide one or more alert notifications to one or more users regarding at least one of: cleaning statistics, cleaning agent refilling status, power level status, system performance diagnostics, sensor calibration alerts, real-time operational status, and model update alerts.

9. The system as claimed in claim 1, wherein the autonomous cleaning device configured with a radio frequency (RF) unit,
the radio frequency (RF) unit configured to:
connect the autonomous cleaning device to one or more servers for generating the cleaning statistics; and
integrate with Internet of Things (IoT)-based facility management systems for automated scheduling of the autonomous cleaning device and reporting to the one or more users.

10. A method for autonomous artificial intelligence-based (AI-based) cleaning of diverse surfaces, comprising:
generating, by a plurality of sensors, sensor data comprises at least one of: depth perception data, bacterial contamination data, inertial measurement data, and chemical compositions;
obtaining, by a microcontroller unit through a data obtaining subsystem, the sensor data for initiating the cleaning of diverse surfaces;
triggering, by the microcontroller unit through a spatial recognition subsystem, a first actuation unit associated with a rotating arm configured with the visual data capturing unit to capture the first visual data of the diverse surfaces to determine one or more spatial characteristics of the diverse surfaces;
changing, by the microcontroller unit through an adaptative height alteration subsystem, a height of the autonomous cleaning device based on the one or more spatial characteristics through a third actuation unit associated with a plurality of articulated shafts;
regulating, by the microcontroller unit through the adaptative height alteration subsystem, speed of a fourth actuation unit associated with a wheel assembly to move the autonomous cleaning device based on the one or more spatial characteristics;
analysing, by the microcontroller unit through a navigation subsystem, at least one of: the first visual data, the depth perception data, and the infrared (IR) data through at least one of:
one or more predictive motion models, one or more deep reinforcement learning models to generate obstacle avoidance data;
processing, by the microcontroller unit through the navigation subsystem, the obstacle avoidance data and the first visual data through at least one of: one or more path planning models, one or more reinforcement learning models, and one or more motion planning models to generate a road map to navigate the autonomous cleaning device to a targeted surface within the diverse surfaces;
analysing, by the microcontroller unit through a surface scanning subsystem configured with one or more convolutional neural networks (CNNs), the first visual data and the sensor data for determining at least one of: one or more geometrical features and contamination data on the targeted surface;
analysing, by the microcontroller unit through a decontamination subsystem, the contamination data through the one or more convolutional neural networks (CNNs) to determine at least one of: a corresponding cleaning equipment from one or more cleaning equipment and a quantity of a cleaning agent;
analysing, by the microcontroller unit through the decontamination subsystem, the one or more geometrical features through a gyroscopic stabilization mechanism to maintain a consistent contact between the targeted surface and the corresponding cleaning equipment through a fifth actuation unit and a sixth actuation unit associated with the cleaning equipment assembly and the liquid tank respectively;
triggering, by the microcontroller unit through the decontamination subsystem, a seventh actuation unit associated with the liquid tank to dispense the determined quantity of the cleaning agent on to the corresponding cleaning equipment based on the contamination data;
triggering, by the microcontroller unit through the decontamination subsystem, a cleaning equipment actuation unit to initiate a first-round of cleaning in pre-defined sequential patterns on the targeted surface based on the contamination data; and
activating, by the microcontroller unit through an intelligent spot detection subsystem, at least one of: the visual data capturing unit and the one or more chemical sensors to capture second visual data to identify at least one of: particulate accumulation and contaminants on the targeted surface to initiate a second-round cleaning.
